(19) United States
(12) Patent Application Publication
(10) Pub. No.: US 2018/0228470 A1
(43) Pub. Date: Aug. 16, 2018

(54) ENHANCED CHONDROGENESIS IN THE PRESENCE OF MICROBUBBLES AND ULTRASOUND

(71) Applicant: The George Washington University, Washington, DC (US)

(72) Inventors: Mitra ALIABOUZAR, Laurel, MD (US); Kausik SARKAR, Washington, DC (US); Lijie Grace ZHANG, Washington, DC (US)

(73) Assignee: The George Washington University, Washington, DC (US)

(21) Appl. No.: 15/823,064

(22) Filed: Nov. 27, 2017

Related U.S. Application Data
(60) Provisional application No. 62/459,983, filed on Feb. 16, 2017.

(51) Int. Cl. A61B 8/08; C12N 5/077; A61N 7/02

(52) U.S. Cl. CPC A61B 8/481; A61N 2007/0017; A61N 7/02; C12N 5/0655

(57) ABSTRACT

Enhancing chondrogenesis at a desired site by the application of ultrasound in the presence of microbbubles. A biomimetic scaffold may be seeded with stem cells capable of undergoing chondrogenesis and provided to the site and then subjected to ultrasound excitation in the presence of microbubbles.

### CROSS-REFERENCE TO RELATED APPLICATIONS

The present application claims the benefit of U.S. Provisional Application No. 62/459,983, filed Feb. 16, 2017, which is hereby incorporated by reference in its entirety.

### TECHNICAL FIELD

The present disclosure relates to the formation or repair of cartilage, and more particularly relates to enhanced chondrogenesis employing ultrasound and microbubbles as an ultrasound contrast agent.

### BACKGROUND

Over 6 million people visit hospitals due to cartilage damage every year. Cartilage injury leads to arthritis, which involves the erosion of the articulating surfaces of joints, and is the most common disabling human condition affecting 33.6% of adults aged 65 and older in the United States.

Cartilage is an avascular tissue notorious for its complex stratified structure as well as for its very low capacity of self-repair after injury. Existing methods of treatment, such as allografts, autografts and total joint replacement, have been used but may involve complications including donor site morbidity, insufficient donor tissues and infection. Nearly 11% of patients with hip replacements and 8% of those with knee replacement had revision operations in 2003 in the United States due to failed implant surgeries.

### BRIEF DESCRIPTION OF THE DRAWINGS

In order to describe the manner in which the above-recited and other advantages and features of the disclosure can be obtained, a more particular description of the principles briefly described above will be rendered by reference to specific embodiments thereof which are illustrated in the appended drawings. Understanding that these drawings depict only exemplary embodiments of the disclosure and are not therefore to be considered to be limiting of its scope, the principles herein are described and explained with additional specificity and detail through the use of the accompanying drawings in which:

FIG. 1 is an exemplary graph showing a wave function representing acoustic pressure along with its effect on microbubbles;

FIG. 2A is a diagram of an exemplary stereolithography based 3D printer system;

FIG. 2B is a scanning electron microscope micrograph of an exemplary scaffold made up of PEGDA at 100 μm;

FIG. 2C is a scanning electron microscope micrograph of an exemplary scaffold made up of PEGDA at 200 μm;

FIG. 3 is a diagram of an example system to enhance chondrogenesis of cells employing low intensity pulsed ultrasound in the presence of microbubbles;

FIG. 4 is a diagram of a knee being treated to enhance chondrogenesis;

FIG. 5 is a graph illustrating an example microbubble size distribution before and after centrifugation;

FIG. 6 is a graph showing the results of human Mesenchymal stem cells showing no short or long term cytotoxicity;

FIG. 7A is a graph illustrating microbubbles at varying concentrations to determine an optimal concentration;

FIGS. 7B, 7C, and 7D are example images of human Mesenchymal stem cell growth before and after LIPUS stimulation in the presence of microbubbles;

FIG. 8A illustrates glycosaminoglycans production over a three week period with low intensity pulsed ultrasound stimulation in the presence of microbubbles;

FIG. 8B illustrates Type II Collagen synthesis over a three week period with low intensity pulsed ultrasound stimulation in the presence of microbubbles;

FIG. 9A is a graph showing the effect of 3 minute low intensity pulsed ultrasound stimulation at various intensities on human Mesenchymal stem cell proliferation;

FIG. 9B is a graph showing low intensity pulsed ultrasound stimulation at different duty cycles on human Mesenchymal stem cell proliferation;

FIG. 9C is a graph showing the change in human Mesenchymal stem cell proliferation with low intensity pulsed ultrasound frequency; and

FIG. 9D is a graph showing the effect of low intensity pulsed ultrasound excitation time period (mins) on hMSC proliferation in the presence of microbubbles.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure. It should be understood at the outset that although illustrative implementations of one or more embodiments are illustrated below, the disclosed compositions and methods may be implemented using any number of techniques. The disclosure should in no way be limited to the illustrative implementations, drawings, and techniques illustrated herein, but may be modified within the scope of the appended claims along with their full scope of equivalents.

### Overview

Tissue engineering offers novel approaches towards repairing or replacing damaged tissues for the purpose of restoring tissue functionality. Effective tissue regeneration incorporates a viable cell source, biocompatible and mechanically relevant scaffolds, suitable growth factors and mechanical cues. Disclosed herein is a system and method for repairing, generating and/or regenerating cartilage tissue, for enhanced chondrogenesis. This may be conducted in-vitro or in-vivo with human, mammal or animal subjects. The disclosure herein employs low intensity pulsed ultrasound (LIPUS) along with microbubbles (MBs) as an ultrasound contrast agent. The MBs may have a lipid containing outer shell along with an internal core containing a bioinert gas such as a perfluorocarbon, for instance perfluorobutane. The MBs can be administered parenterally, for instance intravenously, to a subject.

A biomimetic scaffold may also be employed on the site formed via three dimensional (3D) printing lithography. The 3D constructs can be formed and placed at the site of a patient needing repair or regeneration of cartilage. Moreover, the scaffold can be seeded with cells which are capable of undergoing chondrogenesis, such as human mesenchymal cells (hMSC's) and so itself may be the site for chondrogenesis. Alternatively, the site for chondrogenesis, whether having the scaffold or not, may use the subject's own cells. The site for chondrogenesis may be any part of the body requiring cartilage repair or regeneration, such as the knee, or may be carried out in vitro or in vivo.

### Ultrasound

Ultrasound offers unique advantages such as being non-invasive, inexpensive and well understood. Ultrasound is widely used for diagnostic and therapeutic purposes. However clinical utilization of LIPUS (such as intensities lower than 1 W/cm²) has been so far limited to bone fracture healing. Note that, in general bone expresses higher ability for healing while cartilage, as mentioned before, has very limited capability for self-repair. As a result, development of novel tissue engineering constructs and stimulation techniques for treatment of cartilage injuries are of pressing interest. Accordingly, disclosed herein is the use of ultrasound treatment, and in particular LIPUS.

Example LIPUS excitation as disclosed herein may include intensities lower than 1 W/cm², including intensities (units in W/cm²) from about 5 to about 400, alternatively 10 to 300, alternatively 30 to 150, and alternatively from 70 to 100, encompassing any value and subset therebetween. The duty cycle may range from about 0.02% to about 80% (i.e., pulse repetition period (PRP)) over 250 μs to 1 s. A particular PRP and duty cycle for use herein may include a PRP of 1 ms (duty cycle of 20%). The excitation period may extend for multiple days, one day, several hours, one hour, up to 10 minutes, or from 1 to 5 minutes, including about 1 minute, about 3 minutes, or about 5 minutes. The frequency may be for instance 1.5 MHz. A particular ultrasound may be 30 mW/cm², duty cycle 20% and 1.5 MHz for one to three minutes. Any suitable ultrasound device may be employed for providing the desired ultrasound exposure for excitation of the MBs. For instance, the ultrasound may be produced by a function generator (such as 33250A, sold by Agilent), amplified by a power amplifier (such as model A-150, by ENI) and emitted from a single element unfocused immersion transducer. Other suitable generators, and amplifiers and transducers may be employed for excitation.

### Microbubbles

Due to the gas compressibility, MBs are highly responsive to ultrasound, and accordingly may be applied as ultrasound contrast agents (UCA). The combination of LIPUS and MBs has not been previously studied for cartilage tissue regeneration. Ultrasound is non-invasive, well-understood and relatively inexpensive.

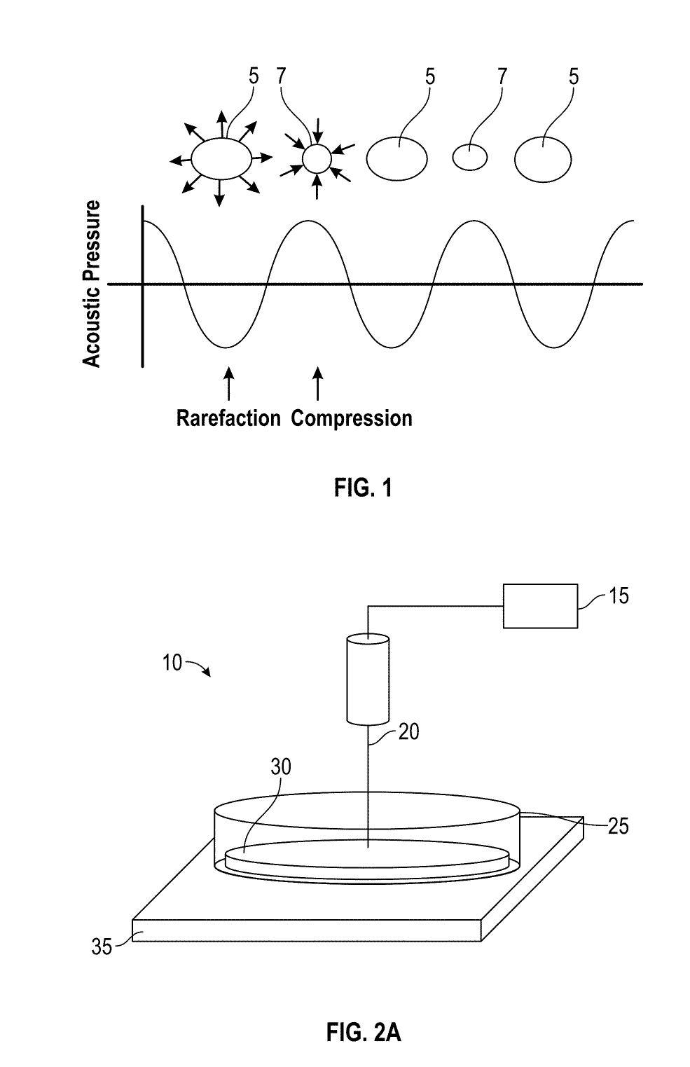

Ultrasound contrast agents such as MBs contain a gas core which makes them compressible. In the presence of ultrasound as disclosed herein, MBs will resonate, rapidly contracting and expanding in response to the pressure changes of the incident wave. As illustrated in FIG. 1, a graph is shown with a wave function representing acoustic pressure. MB's are illustrated above the graph at each peak (highest point above the horizontal axis) and trough (lowest point below the horizontal axis). Rarefaction occurs as shown by the arrow when the wave is below the axis whereas compression occurs as shown by the arrow above the peaks. The expanding MBs 5 are positioned above the graph at the troughs corresponding to rarefaction and the contracting MBs 7 are positioned above the graph corresponding to compression which occurs at the peaks of the wave. The outward facing arrows around the left most expanding MB 5 illustrates expansion of the MB, and the inward facing arrows around the left most contracting MB 7 illustrates the contraction of the MB. It is hypothesized that if these oscillating bubbles are in the vicinity of cells, they exert extra shear forces.

MBs have biocompatible shells, and such shells may contain a lipid. The lipids may have one or more hydrophobic tails and a hydrophophilic head. Particularly suitable lipids include phospholipids. The biocompatibility of the phosopholipids is likely due to the cell membrane of all living cells being primarily composed of phospholipids. The phospholipids disclosed herein may include for instance phosphatidylcholines, phosphatidylglycerols, phosphatidylinositols, phosphatidic acids, and phosphatidylserines.

The phosopholipid may include two or more fatty tails where each, independently from one another, may be include from 8 to 40 carbons, alternatively from 10 to 30, alternatively from 15 to 25, and in particular 18 carbons, and may be branched or straight chained, substituted or unsubstituted, and may be saturated or unsaturated. The phospholipid may include a phosphoryl moiety which may have positively charged counterions such as alkali metals, including Na⁺, K⁺, Li⁺, or alternatively, organic counterions such as NH₄⁺.

The phospholipids as disclosed herein may have a nitrogen containing head group attached to the phosphoryl group. The nitrogen containing head group may be amine, and which may be present as a cation such as ammonium or choline. The counterion to the nitrogen containing cation may be a halide such as chlorine. The head group may be other than a nitrogen group and may include hydroxy, polyhydroxy, alcohol, glycol, glycerol, polyols, ethers, polyethers, or the like bound to the phosphoryl moiety.

Particularly suitable phospholipids include, but are not limited to, 1,2-dipaInnitoyl-sn-glycero-3-phosphatidylcholine (DPPC), 1,2-dipalmitoyl-sn-glycero-3-phosphatidylethanolannine-polyethyleneglycol-2000 (DPPE-PEG-2000) or 1,2-dipalmitoyl-3-trimethylammonium propane (chloride salt; 16:0 TAP).

The inner core of these MBs contain a gas such as atmostpheric air, nitrogen, oxygen, carbon dioxide, hydrogen, an inert gas, perfluorobutane (C4F10, “PFB”) which is also bio inert. The gas may have a low molecular weight so as to be in gaseous form in a human body. In particular perfluorinated gasses may be employed, including perfluoroalkanes (perfluorocarbons) such as perfluoropropane, perflurobutane, or perfluorpentane, as well as perfluoralkenes, and perfluorocycloalkanes. Additional gases include perfluorinated ketones and perfluroinated ethers. Gases for use in MBs which provide high stability in the bloodstream are particularly suitable.

The MBs can be prepared in any container, such as a glass container, by providing a lipid emulsion by dissolving the lipids, for instance in concentrations of 0.75 to 3 mg/mL glycerol (or other alcohol, polyol, or polar solvent). The gaseous head space in the container may be exchanged with the desired gas such as perfluorobutane, or others noted above, and agitating or mixing the solution until MBs are formed.

The MB may be provided in a concentration that is effective to enhance chondrogenesis, and may be provided in a range from about 0.01 to about 5% by volume of a cell media, alternatively from about 0.1% to about 1%, alternatively from about 0.25% to 0.75%, and may be added at about 0.5%. Particular MBs which may be suitably employed include FDA approved MBs as ultrasound contrast agent, such as DEFINITY® MBs.

### Cells

The cells employed herein are capable of undergoing chondrogenesis or otherwise producing cartilage constituents and forming cartilage extra cellular matrix (ECM) at a target site. The cartilage constituents may form cartilage or the components for generating or repairing cartilage. Proteoglycan and type II collagen are two major constituents of the ECM of cartilage tissue, contributing to compressive and tensile properties of cartilage tissue, respectively. Among the constituents that may be formed by the cells include glycosaminoglycans (GAG), total collagen and type II collagen. The cells may be endogenous to the subject, which may be human or animal or other organism, or from external sources. Accordingly, the site, with or without a scaffold, which will be the subject of cartilage growth may be seeded with cells from an external source or may be from the subject itself. The scaffold may be pre-seeded with cells, optionally subject to LIPUS excitation in the presence of MBs, and then placed at the desired site on the subject. In some cases the scaffold is not seeded beforehand, and when placed at the site for desired chondrogenesis, the subject's own cells may arrive at the site via natural processes. The site may then be subjected to LIPUS in the presence of MBs to enhance the chondrogenesis process.

Particularly suitable cells which may be used for seeding and chondrogenesis include human Mesenchymal stem cells (hMSCs) which are abundant and have the potential to differentiate into many cell lines including cartilage and bone. The stimulation technique disclosed herein involving the application of ultrasound and MB's significantly increases at least the amount of GAG, total collagen and type II collagen.

### Scaffold

A biomimetic scaffold may be employed to act as a structure for generating the cartilage. In particular stereolithography three dimensional (3D) printing may be employed to prepare the scaffold. The 3D-printed constructs may be made to match the mechanical, acoustic properties of the native cartilage tissue.

The components used to prepare the scaffold may be bio-compatible polymers, including polyethylene glycol (PEG), polyethylene glycol diacrylate (PEGDA), polyglycolic acid (PGA), polycaprolactone (PCL), polylactic-co-glycolic acid (PLGA), and/or polylactic acid (also referred to as poly(lactic) acid, polylactide, PLA). One or both of the isomers of PLA may be used including poly(L-lactic acid) and poly(D-lactic acid).

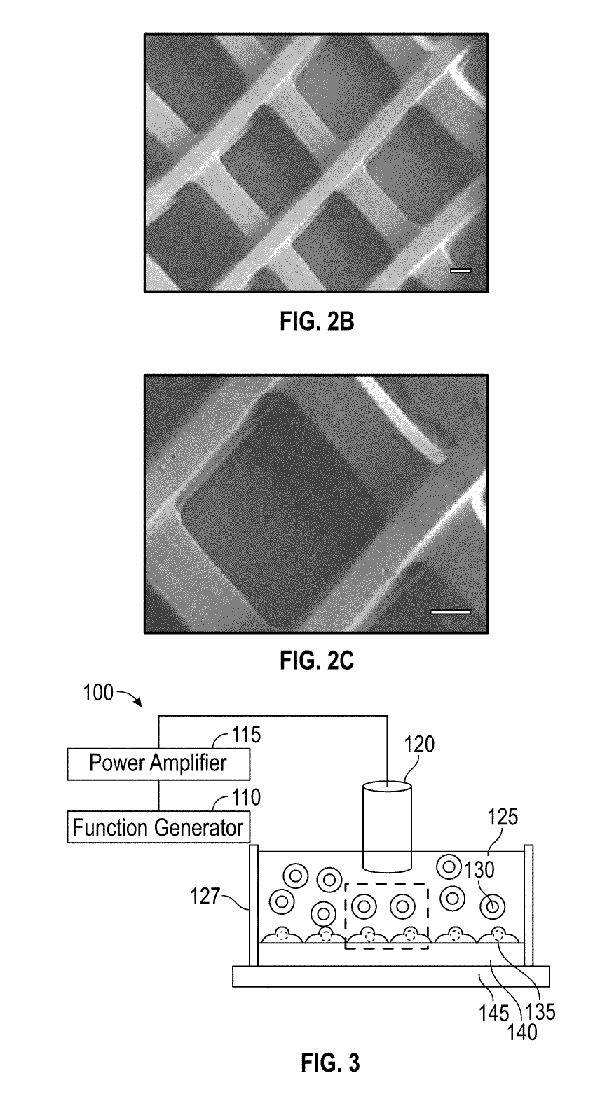

The scaffolds may be porous and have channels of various shapes. The shapes may be polygonal, may have a plurality of sides such as from three to ten, or alternatively from four to eight, may be regular or irregular, and may be quadrilateral, square, or rectangle. The channel shapes may also be circular or elliptical. An exemplary schematic of a stereolithography based 3D printer system 10 is illustrated in FIG. 2A. As shown therein, a ultraviolet (UV) laser source 15 projects an UV beam 20 into a container 25 (such as a petri dish) containing a resin (polymer) 30. A moveable platform 30 is adjusted during the process to form the desired structure of the scaffold. In the illustrated embodiment, scanning electron microscope (SEM) micrographs of an exemplary scaffold made according to a process such as depicted in FIG. 2A and from PEGDA are illustrated in FIGS. 2B and 2C. As illustrated the scaffold in FIGS. 2B and 2C have square channels. FIG. 2A illustrates the scaffold at 100 μm, and FIG. 2B illustrates the scaffold at 200 μm.

### Treatment

Illustrated in FIG. 3 is a diagram of an example system 100 to enhance chondrogenesis of cells employing LIPUS with MBs. In system 100 there is a function generator 110 along with a power amplifier 115 and transducer 120 to project LIPUS into the cell media 125 provided within container 127. The cell media may include necessary nutrients for vitality of the cells as well as MBs 130. In the example embodiment, the MB suspension may be present at about 0.5% (v/v), for instance, if the volume of the cell media in each well is 3 ml then 0.5% of that will be the MB suspension (3 ml of cell media+(3*0.5%) 15 microliters of MB suspension). The scaffold 140 may be seeded with cells 135 prior to introduction into the container 127. The scaffold 140 may be a 3D printed scaffold as disclosed herein. As shown in FIG. 3, the scaffold 140 seeded with cells 135 may be placed toward the bottom of the container 127 and have an acoustic absorber 145 underneath to absorb LIPUS passed through the cell media 125. The seeded scaffold 140 may be considered to be a site having cells capable of undergoing chondrogenesis. The MBs oscillate with the LIPUS pulses in the vicinity of the cells, exerting extra mechanical pressure. The cells chondrogenesis is enhanced as a result resulting in the growth of cartilage or its growth. Accordingly, chondrogenesis may be enhanced in vitro with LIPUS and MBs.

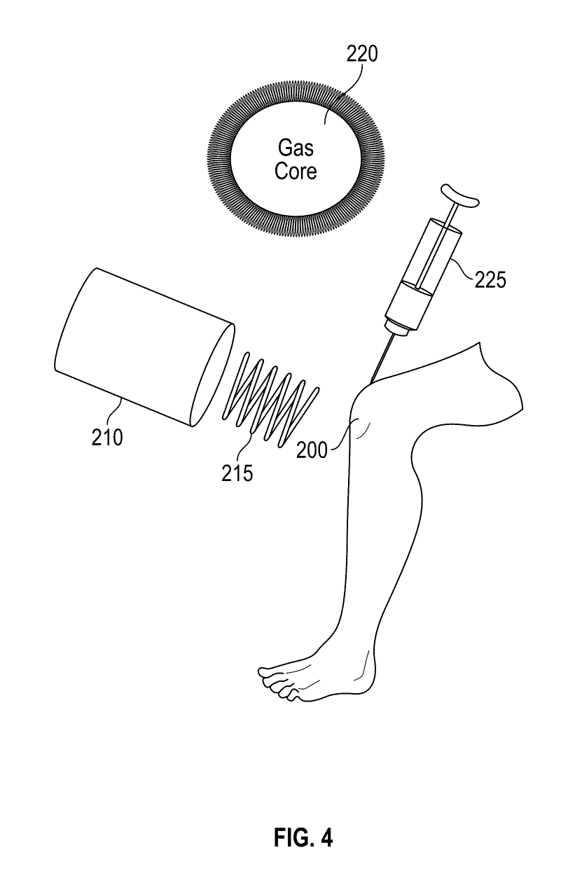

After the scaffold has been seeded and then treated by LIPUS in the presence of MBs, the treated scaffold may be employed in in vivo implantation, for instance implanting into a desired subject, such as a human, mammal or other animal. As illustrated in FIG. 4, the treated scaffold may be placed at the site needing cartilage repair, such as a knee 200. While a knee is shown herein, this may be applied to any place within a subject having or needing cartilage, such as joints, elbows, ankles, hips, or other regions. MBs 220 having a gas core and lipid outer shell may be delivered to the knee 200 by administering an injection of MBs 220 to the subject via a needle 225. These MBs are stable and small enough (for instance having an average diameter of 1.2 micrometers) to pass through the veins and capillaries. The MB dosing here can be for instance about 10 μl/Kg of the subject. The dosing may range from 5 to 15 μl/Kg of the subject, or any effective amount. The MB's will travel to the site, in this case knee 200. Thereafter, a transducer 210 located outside the body may expose the site with the LIPUS.

Alternatively, the scaffold need not be seeded prior to introduction into a subject. The scaffold can be placed at the desired site on the subject (such as the knee in FIG. 4) and subjected to LIPUS and MB as discussed. In such cases, cells endogenous to the subject may be sufficient for chondrogenesis. The cells may naturally be present at the site or travel to the site through the subject's natural processes.

Alternatively, the scaffold may be omitted entirely and need not be placed at the site desired for chondrogenesis at all, and rather, the site either seeded with external hMSC's or other cells (such as other stem cells), or subjected to LIPUS in the presence of MBs without seeding. Accordingly, the site may be simply subjected to LIPUS along with the introduction of MBs (such as in FIG. 4), and chondrogenesis enhanced with the patients own cells and without a scaffold.

To facilitate understanding of the present disclosure, the following examples of certain embodiments are provided, and in no way should the following examples be read to limit, or define, the scope of the disclosure.

### EXAMPLES

### Example 1

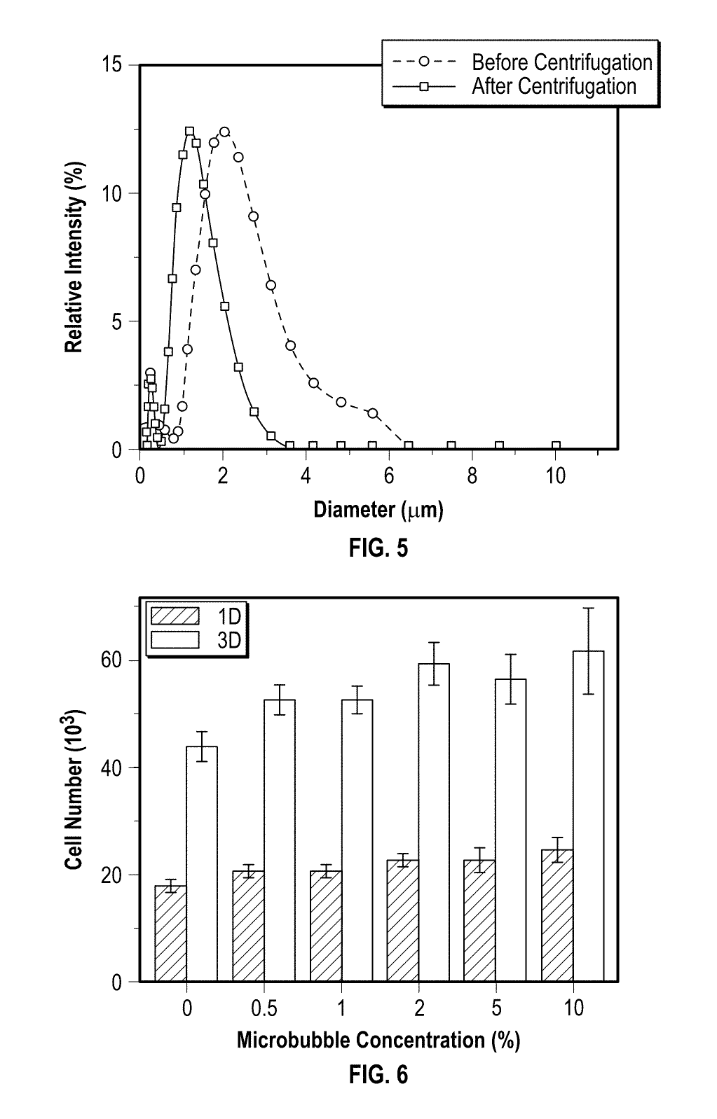

A stock solution of lipid coated MBs were formed by adding a 1.5 ml solution to a 3 ml glass vial. The head space was exchanged with PFB and MBs were formed via mechanical agitation using a vial mixer for 45 seconds. FIG. 5 illustrates the resulting size distribution before and after centrifugation.

### Example 2

In order to assess cell viability, hMSC's were incubated with lipid-coated MBs using 1 day (short term) and 3 day (long term) terms with a number of MB concentrations of 0, 0.5%, 1%, 2%, 5% and 10% (v/v). As illustrated by the increased cell count number for the 3 day period in FIG. 6, the MBs have no short or long term cytotoxicity.

### Example 3

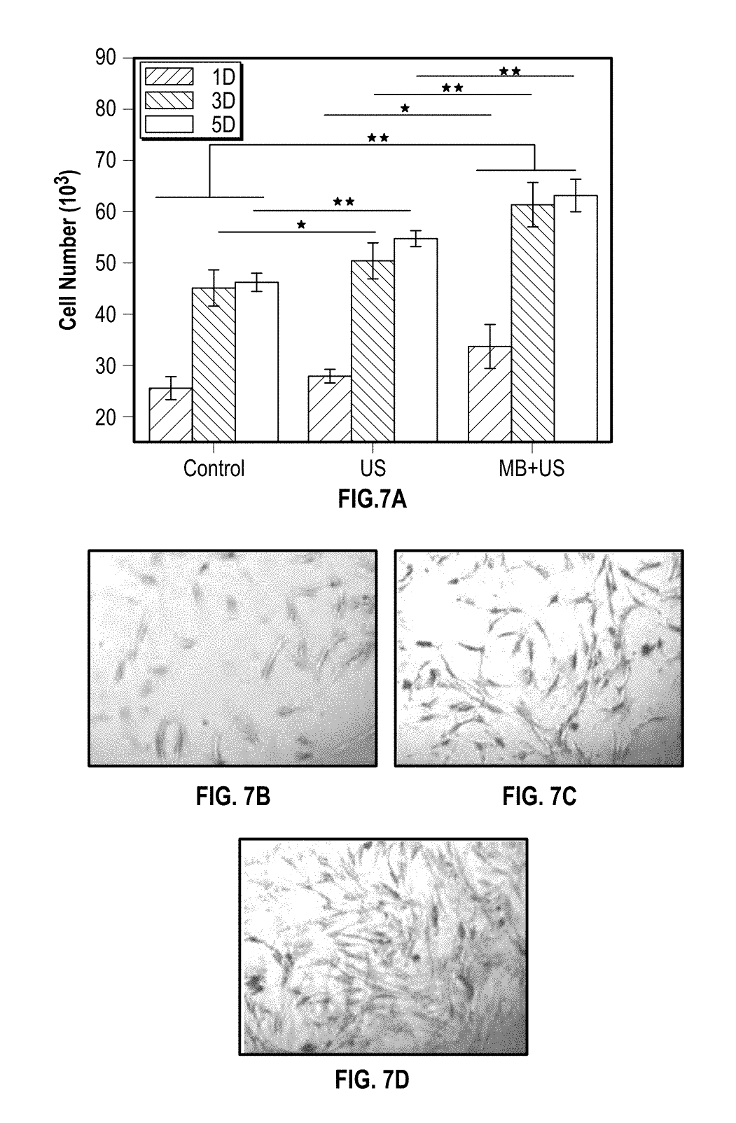

Varying amounts of MB were added to fresh media to test one embodiment of an optimal concentration of MBs. Following the addition of MB, LIPUS (30 mW/cm², duty cycle 20% and 1.5 MHz) was applied for three minutes. As illustrated the graph FIG. 7A, LIPUS with 0.5% (v/v) MB solution resulted in the maximum hMSC proliferation. A subsequent 3-day LIPUS stimulation in the presence of this concentration (0.5% (v/v) led to an almost 40% increase in hMSC proliferation also shown in the graph of FIG. 7A. FIGS. 7B, 7C, and 7D illustrate images of hMSC growth, with FIG. 7B being the control (no LIPUS or MB), FIG. 7C having only LIPUS stimulation, and FIG. 7D LIPUS with 0.5% (v/v) MB solution.

### Example 4

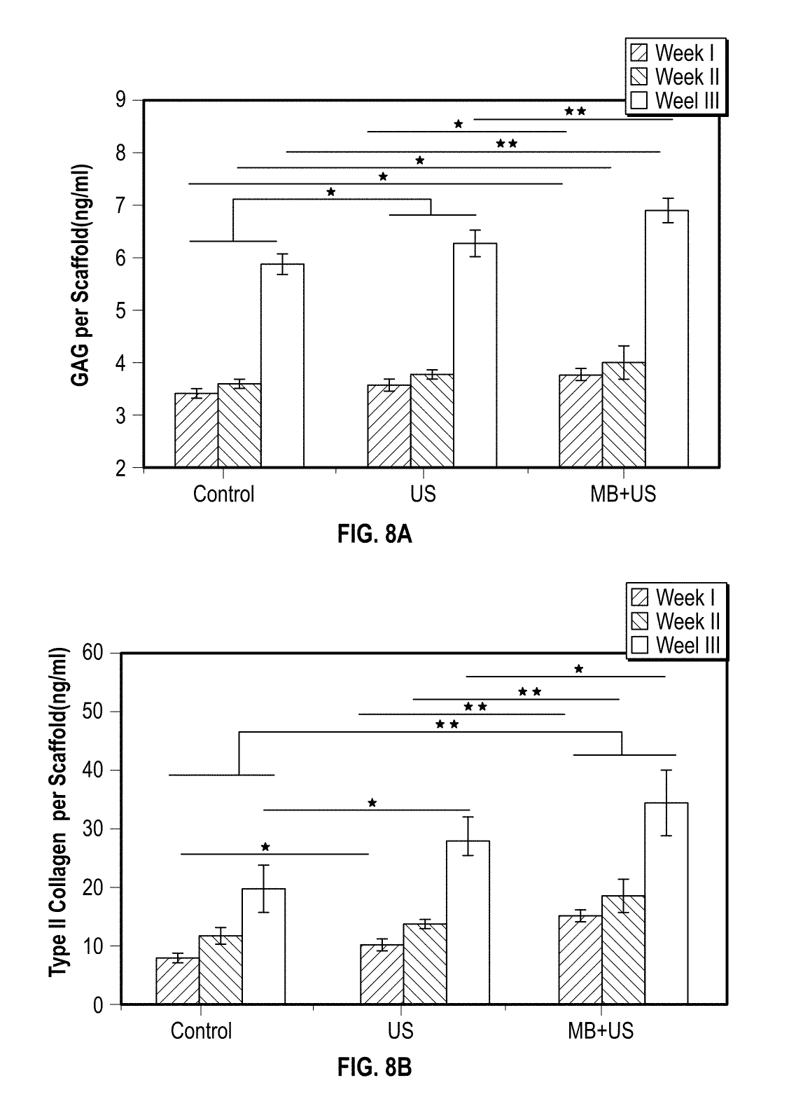

FIG. 8A illustrates a three week LIPUS stimulation in the presence of MBs. As shown, there is enhanced GAG production, up 17% as compared to the control of no LIPUS and LIPUS only. FIG. 8B illustrates a 78% increase in Type II Collagen synthesis after three weeks of LIPUS and MB treatment. LIPUS alone resulted in a 44% increase compared to the control having no LIPUS or MB.

### Example 5

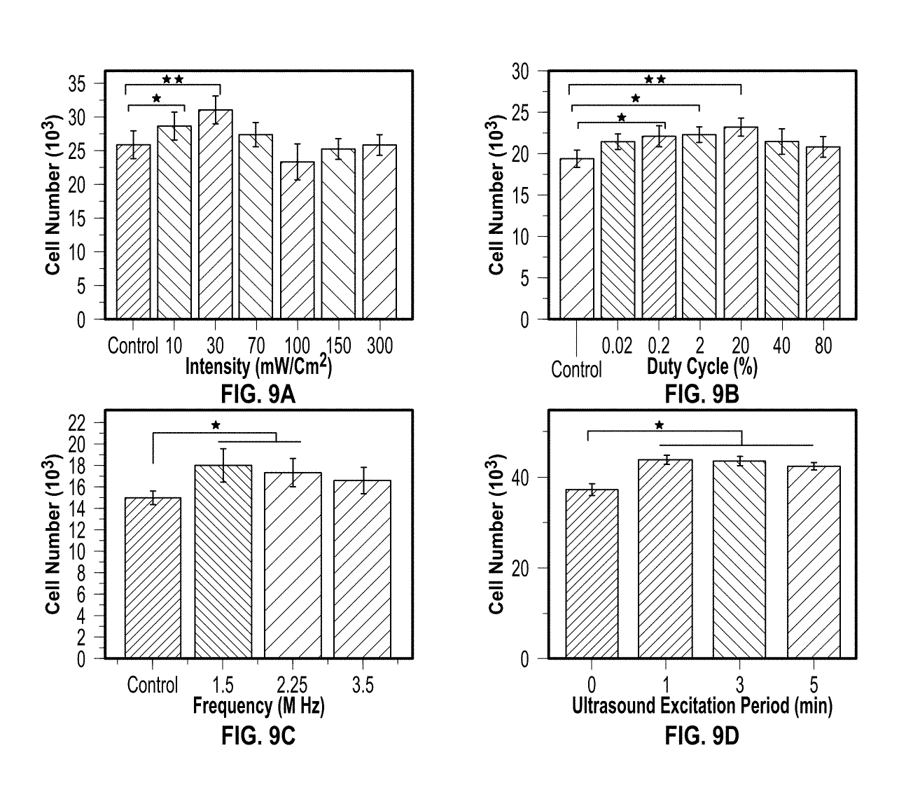

Example 5 illustrates testing to determine an optimized set of acoustic parameters on the proliferation rate of hMSCs in the presence of MB. FIG. 9A illustrates the effect of 3 minute LIPUS stimulation at various intensities in the presence of 0.5% (v/v) MB solution, with the optimal intensity in this case indicated to be 30 mW/cm². FIG. 9B shows the change in proliferation rate of hMSC at different duty cycles in the presence of 0.5% (v/v) MB on day 1, showing an optimal duty cycle in this instance of 20%. FIG. 9C illustrates the change in proliferation of hMSCs with LIPUS frequency in the presence of 0.5 (v/v) MB on day 1, showing optimal frequencies in this instance of 1.5 and 2.25 MHz. FIG. 9D illustrates the effect of LIPUS excitation period on hMSC proliferation on day 1 in the presence of 0.5% (v/v) MB solution. Time 0 shows the control group, as it had not yet been subject to LIPUS excitation.

We claim:

1. A method for enhancing chondrogenesis comprising:
delivering an ultrasound contrast agent to a site containing cells which are capable of undergoing chondrogenesis; and
subjecting the site to an ultrasound treatment,
whereby cartilage constituents are formed from the cells.

2. The method of claim 1, wherein the ultrasound contrast agent is microbubbles.

3. The method of claim 2, wherein the microbubbles have a biocompatible outer shell.

4. The method of claim 3, wherein the outer shell contains a lipid.

5. The method of claim 2, wherein the microbubbles contain a gaseous fluorocarbon.

6. The method of claim 2, further comprising delivering the microbubbles to the site via parenteral administration.

7. The method of claim 1, wherein the cartilage constituents are selected from the group consisting of glycosaminoglycans (GAGs), total collagen, type II collagen, and mixtures thereof.

8. The method of claim 1, wherein the ultrasound treatment is low intensity pulsed ultrasound.

9. The method of claim 1, wherein the site contains a biomimetic scaffold.

10. The method of claim 9, wherein the biomimetic scaffold is formed by three dimensional printing lithography.

11. The method of claim 9, wherein the biomimetic scaffold comprises one or more of polylactic acid (PLA), polyethylene glycol (PEG), polyethylene glycol diacrylate (PEGDA), polyglycolic acid (PGA), and polylactic-co-glycolic acid (PLGA).

12. The method of claim 9, further comprising seeding the biomimetic scaffold with human mesenchymal cells.

13. The method of claim 9, further comprising implanting the biomimetic scaffold in a human subject.

14. The method of claim 1, wherein the site is located within a human subject.

15. The method of claim 1, wherein the method is conducted in-vitro.

16. A system for enhancing chondrogenesis comprising:
a microbubble ultrasound contrast agent present at a site containing cells which are capable of undergoing chondrogenesis; and
a device configured to deliver an ultrasound treatment to the site;
whereby cartilage constituents form from the cells upon receiving the ultrasound treatment.

17. The system of claim 16, wherein the microbubbles have an outer shell containing lipids.

18. The system of claim 16, wherein the microbubbles contain a gaseous fluorocarbon.

19. The system of claim 16, wherein the site contains a biomimetic scaffold formed by three dimensional printing lithography.

20. The system of claim 19, wherein the biomimetic scaffold is seeded with human Mesenchymal cells.
